(19) United States
(12) Patent Application Publication
(10) Pub. No.: US 2019/0239806 A1
(43) Pub. Date: Aug. 8, 2019

(54) WEARABLE RESPIRATORY INDUCTANCE PLETHYSMOGRAPHY DEVICE AND METHOD FOR RESPIRATORY ACTIVITY ANALYSIS

(71) Applicant: Carre Technologies Inc., Montreal (CA)

(72) Inventors: Jean-Francois ROY, Montreal (CA); Pierre-Alexandre FOURNIER, Montreal (CA); Charles ROBILLARD, Saint-Lazare (CA); Robert CORRIVEAU, Saint-Bruno (CA); Simon DUBEAU, Montreal (CA); Antoine GAGNE-TURCOTTE, Montreal (CA); David KHOUYA, St-Eustache (CA)

(21) Appl. No.: 16/388,763

(22) Filed: Apr. 18, 2019

Related U.S. Application Data
(63) Continuation-in-part of application No. 14/955,749, filed on Dec. 1, 2015, now abandoned.

(30) Foreign Application Priority Data
Dec. 2, 2014 (CA) 2,872,754
Jul. 9, 2015 (CA) 2,896,498

(51) Int. Cl. A61B 5/00; A61B 5/0205; A61B 5/01; A61B 5/11

(52) U.S. Cl. CPC A61B 5/6804; A61B 5/02055; A61B 5/01; A61B 5/0002; A61B 5/0816; A61B 5/4803; A61B 5/1118; A61B 5/7221; A61B 5/4818

(57) ABSTRACT

It is described a system and a method for respiratory activity analysis comprising the use of Respiratory Inductance Plethysmography (RIP). In particular, a wearable system for extracting physiological parameters of a person by measuring at least one plethysmographic signal is disclosed. The system comprises: a wearable garment fitting a body part of the person; at least one wire supported by or embedded into the garment, each wire forming a loop around the body part when the person wears the garment for measuring a plethysmographic signal; and an electronic device supported by or fixed on the garment and including a Colpitts oscillator connected to each wire loop, wherein the Colpitts oscillator has an optimal frequency band from 1 MHz to 15 MHz for extracting the plethysmographic signal measured by each wire, the electronic device converting analog information measured by the Colpitts oscillator into digital analyzable information.

### CROSS-REFERENCE TO RELATED APPLICATIONS

The present patent application claims the benefits of priority of commonly assigned Canadian Patent Application no. 2,872,754, entitled “LOW-POWER RESPIRATORY INDUCTANCE PLETHYSMOGRAPHY DEVICE, INTELLIGENT GARMENTS OR WEARABLE ITEMS EQUIPPED THEREWITH AND A METHOD FOR RESPIRATORY ACTIVITY ANALYSIS” and filed at the Canadian Intellectual Property Office on Dec. 2, 2014; and Canadian Patent Application no. 2,896,498, entitled “WEARABLE RESPIRATORY INDUCTANCE PLETHYSMOGRAPHY DEVICE AND METHOD FOR RESPIRATORY ACTIVITY ANALYSIS”, filed at the Canadian Intellectual Property Office on Jul. 9, 2015 and opened to public inspection in advance on Oct. 21, 2015. The present patent application is a continuation-in-part of the U.S. patent application Ser. No. 14/955,749 entitled “WEARABLE RESPIRATORY INDUCTANCE PLETHYSMOGRAPHY DEVICE AND METHOD FOR RESPIRATORY ACTIVITY ANALYSIS”, filed at the United States Patent and Trademark Office on Dec. 1, 2015. The content of these applications is incorporated herewith by reference.

### FIELD OF THE INVENTION

The present invention relates to the field of ambulatory and non-invasive monitoring of an individual's physiological parameters. In particular, it is described a system and a method for respiratory activity analysis comprising the use of Respiratory Inductance Plethysmography (RIP) sensor using an optimal Colpitts oscillator configuration for an efficient human body measurement. The system can be a garment or other wearable item.

### BACKGROUND

Physiological sensors have long been known and widely used for medical and health related applications. Various physiological sensors embedded in textile or garments, sometimes called portable or wearable sensors, have been described before in publications and patents (Portable Blood Pressure in U.S. Pat. No. 4,889,132; Portable device for sensing cardiac function in U.S. Pat. No. 4,928,690; Heart rate monitor in garment in U.S. Pat. No. 7,680,523 B2). The term “wearable sensors” is now commonly used to describe a variety of body-worn sensors to monitor activity, environmental data, body signals, biometrics, health related signals, and other types of data. Garment may include a stretchable harness such as in U.S. Pat. No. 8,818,478 B2.

As used herein, “plethysmography”, and its derivative words, is the measurement of changes in volume within an organ or whole body, or a cross-sectional area of the body when the body's is constant in height. “Inductive plethysmography” is a plethysmographic measurement based on determination of an inductance or a mutual inductance. A “plethysmographic signal” is a signal generated by plethysmography, and specifically by inductive plethysmography. The cross-sectional area of the body measured by a plethysmograph may include, singly or in combination, the chest, abdomen, neck, or arm.

The inductance sensor may be as simple as a conductive loop wrapped around the body cross-section. The loop is attached to a close-fitting garment that expands and contracts with the body cross-section. As the body cross-section expands and contracts, the area enclosed by the loop also expands and contracts thereby changing the inductance of the loop. The inductance change of the loop may be converted to an electrical signal using methods known to one of skill in the electrical art.

If the loop is placed around the chest, the changes in the loop inductance may be correlated to respiration volumes. For example, U.S. Pat. No. 4,308,872 issued Jan. 5, 1982 and titled “Method and Apparatus for Monitoring Respiration,” discloses a method and apparatus for monitoring respiration volumes by measuring variations in the patient's chest cross sectional area.

Respiratory Inductive Plethysmography (RIP) is based on the analysis of the movement of a cross-section of the human torso with a low-resistance conductive loop using conductive textile or knitted warn, wire within an elastic band or braid, a loose wire within a textile tunnel or any conductive material in a configuration that makes it extensible. The extensibility is needed to follow the body as it changes shape due to breathing, movement, or other activities that can modify the body shape and volume.

Many patents and articles mention methods to use RIP sensors such as “Development of a respiratory inductive plethysmography module supporting multiple sensors for wearable systems” by Zhang Z, et al., Sensors 2012; 12, 13167-13184. It is hard to obtain good percentage of effective data as stated at page 23 of the article entitled “A Wearable Respiration Monitoring System Based on Digital Respiratory Inductive Plethysmography”, Bulletin of Advanced Technology Research, Vol. 3, No. 9/Sep. 2009, where only 83% of effectiveness is achieved.

Many types of oscillators have been proposed for RIP sensing and used with different configurations. Noise and artifacts due to movement or other causes are common when RIP sensing is used in a garment or other wearable item. The system must be designed to tolerate noise and artifacts and be able to filter many of them to provide accurate breathing measurements.

Using data from one or many RIP sensors, analysis can provide major metrics such as Respiratory Rate, Tidal Volume and Minute ventilation, Fractional inspiratory time (T inhale, T exhale), and other information about the physiological and psychological state of the person or animal wearing the garment or the wearable item.

Determining signal quality and data quality for wearable sensors is very challenging. The assessment of signal and data quality is an important part of many high-level analysis algorithms, visual presentation of the data, and interpretation of the data in general.

### SUMMARY OF THE INVENTION

The invention is first directed to a wearable system for extracting physiological parameters of a person by measuring at least one plethysmographic signal. The system comprises:

a wearable garment fitting a body portion of the person;

at least one wire supported by or embedded into the garment, each wire forming a loop around the body part when the person wears the garment for measuring a plethysmographic signal; and

an electronic device supported by or fixed on the garment and including a Colpitts oscillator connected to each wire loop;

wherein the Colpitts oscillator has an optimal frequency band from 1 MHz to 15 MHz for extracting the plethysmographic signal measured by each wire, the electronic device converting analog information measured by the Colpitts oscillator into digital analyzable information. In some other embodiments, the optimal frequency band of the Colpitts oscillator may range from 1.6 MHz to 15 MHz.

The invention is also directed to a method for extracting physiological parameters of a person, the method comprising the steps of:
a) providing a wearable garment, the garment fitting a body portion of the person;
b) measuring at least one plethysmographic signal using at least one wire supported by or embedded into the garment, each wire forming a loop around the body part;
c) extracting the plethysmographic signal measured by each wire using an electronic device supported by the garment, the electronic device including a Colpitts oscillator connected to each wire and having an optimal frequency band from 1 MHz to 15 MHz; and
d) converting analog information measured by the Colpitts oscillator into digital analyzable information.

The invention is further directed to the use of the wearable system as disclosed herein, for extracting physiological parameters of a person by measuring at least one plethysmographic signal. Preferably, the physiological parameters extracted by the system are breathing metrics selected from the group consisting of respiratory rate, tidal volume, minute ventilation and fractional inspiratory time.

The invention is further directed to the use of the wearable system as disclosed herein, for detecting and characterizing physical conditions selected from the group consisting of talking, laughing, crying, hiccups, coughing, asthma, apnea, sleep apnea, stress related apnea, relaxation exercise, breathing cycle symmetry, and pulmonary diseases.

The invention is yet further directed to the use of the wearable system as disclosed herein, for detecting and characterizing heart activities selected from the group consisting of heart rate, body movements and activities. Preferably, the body activities are walking and running.

When the user put the garment on, such as a shirt or T-shirt, the wire loops (also named RIP sensors) are then placed around the user body. The garment minimizes the variation in the positioning of the RIP sensor(s) for a better accuracy and repeatability.

Once the low-powered electronic device in connected to the shirt, the Colpitts oscillator circuit is activated to begin the measurement, it measures the area surrounded by the RIP sensor, like a slice of the body. When the user breathes, the sensor move and the area to measure change, by doing so the oscillator circuit change slightly his oscillation frequency reflecting the impedance changes.

Garments, such as shirts, from a complete size set will all have a different inductance with the same oscillator circuit. The electronic device measures main frequency and the delta frequency from the oscillator to estimate the breathing rate, amplitude and volume.

Advantageously, the garment is easy to put allowing to precisely place the sensors providing reliability and accuracy of the Colpitts even for small movement. The garment does not hinder the movements of the person wearing it while providing excellent quality measurements of biometric signals.

In another aspect of the invention, a wearable system for extracting physiological parameters of a person by measuring at least one plethysmographic signal is provided. The system comprises a wearable garment configured to fit a body portion of the person, at least one wire supported by or embedded into the garment, each wire configured to form a loop around the body part when the person wears the garment for measuring a plethysmographic signal, an electronic device supported by or fixed on the garment and including a Colpitts oscillator connected to each wire loop, a temperature sensor configured to measure temperature of the electronic device, wherein the oscillator is configured to extract the plethysmographic signal measured by each wire at a predetermined frequency, the electronic device converting analog information measured by the oscillator into digital analyzable information and wherein the measured temperature of the electronic device is used to calculate a corrected plethysmographic signal from the extracted plethysmographic signal.

The oscillator may be a Colpitts oscillator. The Colpitts oscillator may have an optimal frequency band from 1 MHz to 15 MHz for extracting the plethysmographic signal. The frequency of the Colpitts oscillator may be about 4.3 MHz. or may be about 5.4 MHz.

The at least one wire may have an inductance varying between 76 nH and 4.4 μH. The temperature sensor may have a precision of about 0.1 degree Celsius.

In yet another aspect of the invention, a method for extracting physiological parameters of a person is provided. The method comprises providing a wearable garment, the wearable garment fitting a body portion of the person, measuring at least one plethysmographic signal using at least one wire supported by or embedded into the garment, each wire forming a loop around the body part, extracting the plethysmographic signal measured by each wire using an electronic device supported by the garment, the electronic device comprising a oscillator connected to each wire;

converting analog information measured by the Colpitts oscillator into digital analyzable information, measuring the temperature of the electronic device and calculating a corrected plethysmographic signal from extracted plethysmographic signal using the measured temperature of the electronic device.

The calculation of the corrected plethysmographic signal at a body portion having a variable volume of the person may use the following equation:

BodyPortion_{corrected}(t)=BodyPortion(t)+(tempe(t)−tempe_{ref})*k_{freq-bodyPortion}
Where: Tempe_{ref} is a reference temperature and where k_{—freq} is a factor which varies based on the frequency of oscillation.

The body portion may be selected in any of the thoracic portion, the abdominal portion or arm portion of the person.

In a further aspect of the invention, a method for calibrating extraction of physiological parameters of a person is provided. The method comprises the person wearing a garment comprising at least one wire supported by or embedded into the garment, each wire being configured to form a loop around the body part when the person wears the garment for measuring a plethysmographic signal, extracting the plethysmographic signal measured by each wire using an electronic device supported by the garment, the electronic device comprising a oscillator connected to each wire, measuring data regarding vital capacity of the person by taking a plurality of measurements, storing the vital capacity measurements in a data storage in communication with the electronic device, calculating vital capacity coefficient of the person at the time of acquisition of the measurements using the vital capacity measurements, calculating a respiration coefficient parameter based on the vital coefficient of the person and calculating a corrected plethysmographic signal by multiplying by the respiration coefficient parameter with the extracted plethysmographic signal.

The measuring of the vital capacity of the person may use a spirometer. The data regarding vital capacity may comprise any one of breathing rate, minute ventilation or tidal volume.

The method may further comprise disconnecting the data storage from the electronic device prior to the calculating vital capacity coefficient.

The calculation of the corrected plethysmographic signal may use the following equations:

VC_{ref}=VC*kᵣₑₛₚ

VE_{calibrated}(t)=VE_{raw}(t)*kᵣₑₛₚ

where VE is the minute ventilation, VC is the vital capacity value, kresp is the respiration coefficient parameter.

### BRIEF DESCRIPTION OF THE DRAWINGS

The description makes reference to the accompanying drawings wherein like reference numerals refer to like parts throughout the several views and wherein:

FIG. 1 is a diagram the amplitude versus the frequency and the current for the same frequency for a Colpitts oscillator showing the defined optimal frequency range of the Colpitts oscillator when measured around a human body.

FIG. 2 is a high level diagram showing how a battery power Colpitts oscillator can be connected to a garment to do signal acquisition. FIG. 2 also shows the digital signal processing (DSP) that could be performed to provide useful data statistics and filtered signals.

FIG. 3 as an example of the state machine for algorithm based on two RIP sensors data to extract the breathing rate, the minute ventilation and the tidal volume.

FIG. 4 is an example of how the wearable garment artifacts can be filtered out.

FIG. 5 show a Smith chart result of the RIP sensor stimulated between 1 MHz and 10 MHz showing the good linearity response of the Colpitts oscillator.

FIG. 6 shows garments that use the present system to connect textiles sensors for heart and breathing monitoring to an electronic device with an accelerometer and a BLUETOOTH® wireless connection, the BLUETOOTH® wireless connection being a type of wireless technology for exchanging data over short distance. The electronic device also contains analog and digital filters and amplifiers, a microprocessor device, solid-state memory storage, sensor circuits, power management circuits, buttons, and other circuits.

FIG. 7 shows an example of a garment that includes RIP sensors, electrical, thermal, and optical sensors for cardiac monitoring, breathing monitoring, blood pressure monitoring, skin temperature and core temperature monitoring to an electronic device with position and movement sensors and a wireless data connection.

FIG. 8 shows graphs of plethysmographic signals (corrected and raw) and of the temperature as a function of the time showing an increase in temperature as the oscillation frequency of the sensor decreases.

FIG. 9 is a diagram showing an embodiment of a wearable system comprising a loop connected to an electronic device having a temperature sensor in accordance with the principles of the present invention.

### DETAILED DESCRIPTION OF PREFERRED EMBODIMENTS

The foregoing and other features of the present invention will become more apparent upon reading of the following non-restrictive description of examples of implementation thereof, given by way of illustration only with reference to the accompanying drawings.

Low power sensing is a domain with many technological challenges for designers and manufacturers of e-textile solutions, intelligent garments, wearable sensors, and multi-parameter wearable connected personal monitoring systems.

As aforesaid, the present invention first concerns a wearable system for extracting physiological parameters of a person by measuring at least one plethysmographic signal. The system first comprises a wearable garment fitting a body part of the person.

By “garment”, it is understood any sort of garment or clothing that can be worn by a person. The garment when worn should fit sufficiently the body of the person to be in close contact with the body to follow the movement of the body. Adjusted T-shirt is particularly adapted but any other sort of clothing can be used as long as it fits the body. A belt strapped or a tube around the torso can also be used instead of a T-shirt. The garment can be made of any kinds of fabrics. Preferably, the wearable system according to the invention is washable.

The system also comprises at least one wire supported by or embedded into the garment. Each wire forms a loop around the body part, when the person wears the garment for measuring a plethysmographic signal.

By “supported”, it is to be understood that the RIP wire is the RIP wire loop could be woven, knitted, laminated, glued, stitched or even soldered to the garment. By “embedded”, it is to be understood that the wire loop is enclosed in a protective element supported by the garment. It can be a overstitching into the fabric or a guiding portion as detailed below.

As aforesaid, by “plethysmography”, and its derivative words, is meant the measurement of a cross-sectional area of a body when the body is constant or almost constant in height. By “Inductive plethysmography”, it is meant a plethysmographic measurement based on determination of an inductance or a mutual inductance. By “plethysmographic signal”, it is meant a signal generated by plethysmography, and specifically by inductive plethysmography. The cross-sectional area of the body measured by a plethysmograph may include, singly or in combination, the chest, abdomen, neck, or arm.

The system also comprises a low-powered electronic device supported by or fixed on the garment. The device can be attached to the garment, embedded into the garment such in an open or close pocket thereof. The device includes a Colpitts oscillator connected to each wire loop. The Colpitts oscillator was invented in 1918 by Edwin Colpitts, and reference can be made to U.S. Pat. No. 1,624,537.

A Colpitts oscillator is one of a number of designs for LC oscillators, electronic oscillators that use a combination of inductors (L) and capacitors (C) to produce an oscillation at a certain frequency. The distinguishing feature of the Colpitts oscillator is that the feedback for the active device is taken from a voltage divider made of two capacitors in series across the inductor. A change in the cross section of the body measured by the RIP sensor causes the Colpitts oscillator to change its oscillating frequency. A digital and/or analog electronic circuit is used to measure the frequency, the change in frequency, and/or the rate of change of the frequency of the Colpitts oscillator.

The Colpitts oscillator of the system according to the present invention has an optimal frequency band from 1 MHz to 15 MHz in order to extract the plethysmographic signal measured by each wire. The electronic device then converts analog information measured by the Colpitts oscillator into digital analyzable information.

According to a preferred embodiment, the system may further comprise at least one connector embedded into the garment for connecting the Colpitts oscillator to each wire loop. Any sorts of connector know in the art for this application can be used, such as the one developed and patented by the application with Canadian patent no. CA 2,867,205, the content of which is incorporated herein by reference.

According to a preferred embodiment, the garment may comprise at least one guiding portion embedded into the garment. Since each guiding portion is adapted for receiving and maintaining the wires in a predetermined position around the body portion, the number of guiding portion depends on the numbers of wire loops present in the system. The guiding portion can be of any kind known in the art, such as an overstitching in the fabric of the garment.

According to a preferred embodiment, when the body portion is the torso of the person wearing the garment, the system may then comprise a first loop of wire placed around a thoracic section of the torso and a second loop of said wires being placed around an abdominal section of the person; allowing as such to measure the breathing frequency and/or frequency change of the person. Each wire loop is preferably constructed using a conductive material in a configuration that makes the garment extensible textile that fits the wearer body.

According to a preferred embodiment, the system may further comprise a power source or generator for powering the Colpitts oscillator and electronic device. The power source may be external and adapted to be worn by the user, such as in a pocket, or embedded into the garment. More preferably, the power source is embedded in a section of the garment, such as a pocket or an overstitching. The power source can be a battery or any sort of power source adapted to power the electronic device and Colpitts oscillator. Energy harvesting or scavenging systems known in the art can be also used to provide power, such as those using Peltier effect.

According to a preferred embodiment, the Colpitts oscillator is adapted to be turned on and off a plurality of times per second according to a frequency sampling to extend the power life of the power source.

According to a preferred embodiment, the low-powered electronic device is a digital processing device for converting analog information into digital information by applying at least one algorithm to analyze the information. Preferably, the low-powered electronic device may be in communication with a smart phone or a computer using a wireless connection, such as but not limited to a BLUETOOTH® connection, the BLUETOOTH® wireless connection being a type of wireless technology for exchanging data over short distance.

According to a preferred embodiment, the system may further comprise at least one sensor supported or embedded into the garment. Any sensors known in the art for measuring body temperature, blood pressure and/or heart beat frequency can be used.

According to a preferred embodiment, the physiological parameters extracted by the system may be breathing metrics such as, but not limited to, respiratory rate, tidal volume, minute ventilation and fractional inspiratory time.

According to a preferred embodiment, the system may also provide metrics to detect and characterize physical conditions such as, but not limited to, talking, laughing, crying, hiccups, coughing, asthma, apnea, sleep apnea, stress related apnea, relaxation exercise, breathing cycle symmetry, and pulmonary diseases. The system may also provide metrics to detect and characterize heart activities such as, but not limited to, heart rate, body movements and activities, such as, but not limited to walking and running.

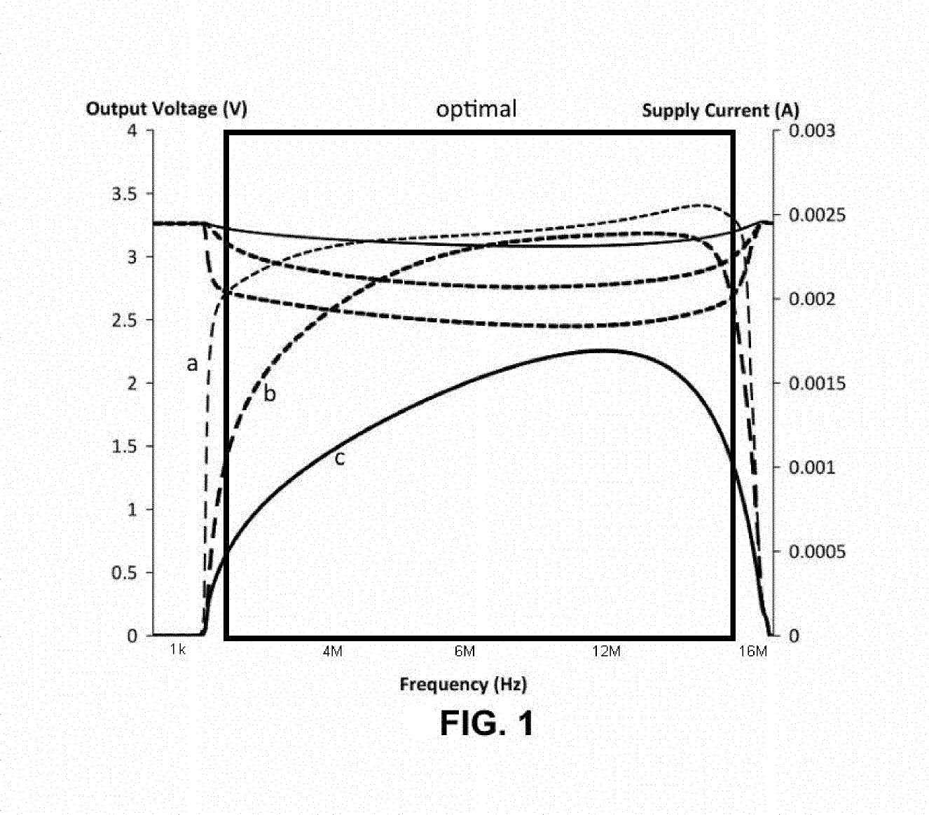

FIG. 1 is a diagram showing the amplitude versus the frequency and the current for the same frequency range for a Colpitts oscillator. An optimal frequency range has been determined and implemented for the impedance loop. This range covers but is not restricted to the frequency band from 1 MHz to 15 MHz. This frequency range has been found to be optimal for the human body composition. The frequency is optimal for maximum precision for a garment or object equipped therewith. The figure shows 3 simulations results with different RIP loop inductance values in the valid range for torso measurements: curves dot-line (a=1.8 μH or microhenry), double-line (b=2 μH) and the plain=−line (c=2.2 μH).

In a typically embodiment, the at least one wire has an inductance varying between 76 nH and 4.4 μH. The wired loop has a low inductance value allowing using of the garment on different living being ranging from 76 nH for a premature baby (3 cm body radius) to 3.6 μH for the largest known human (50 cm body radius—as an example, see https://en.wikipedia.org/wiki/Walter_Hudson_%281944% E2%80%931991%29).

The theoretical inductance of a wire loop may be calculated using the following equation:

$${L}_{circle} \approx {N}^{2} R {μ}_{0} {μ}_{r} [ ln ( \frac{8 R}{a} ) - 2 ]$$

where N is the number of loops, R is the radius of the wire, μr the relative permeability of the medium and L is the inductance (see https://technick.net/tools/inductance-calculator/circular-loop/?aiocp_dp=util_inductance_circle).

The equation for calculating the theoretical inductance does not consider the inductance of the wire itself. In some cases, such inductance may represent about 30 to 70% of the total measured inductance. Thus, the maximum value of the range of inductances for the wired is 4.4 μH to comprise such inductance from the wire itself.

As inductance of the wire loop is low, the wire loop in the garment has relatively high resistance (approx. 1-2 Ohms). In normal Colpitts oscillator design, the supported resistance is between 1.7-100 mOhms. In comparison, the wire loop has a low Q (quality factor) and makes it more difficult to obtain an oscillation with the circuit. As an example, for Q=wL/R, at 5 MHz, Q is around 5. Thus, Colpitts oscillators do not provide reliable results when used with lower inductance loop at such high resistance values thus one skilled in the art typically uses higher inductance loop.

When selecting frequencies of use of the Colpitts oscillator, the low inductance value makes it hard to oscillate at high frequencies (e.g. 500 MHz), for many reasons, one simple reason being it may require capacitors below 0.1 pF, which are not easily available in the industry. As an example, such capacitor is the smallest value available from many manufacturers. Also, capacitor values shall be equal or above 1 pF as larger tolerance on these small capacitors makes it hard to precisely tune the frequency.

Also, using a Colpitts below 1 MHz instead of 1.5-15 MHz would require more power, which is not suitable for a wearable system running on a small power source. Low frequencies also reduce resolution in respiratory measurements. As an example, as an amplitude of ˜50 units for normal breathing at ˜5 MHz is typically obtained with the system, a frequency below 1 MHz would provide an amplitude of less than 10 units, which is not enough resolution to display breathing patterns in a useful manner.

As known prior art systems use low frequency to improve reliability, repeatability and accuracy of the system, the Applicant submits that it would be counter intuitive for a person skilled in the art to use higher frequencies.

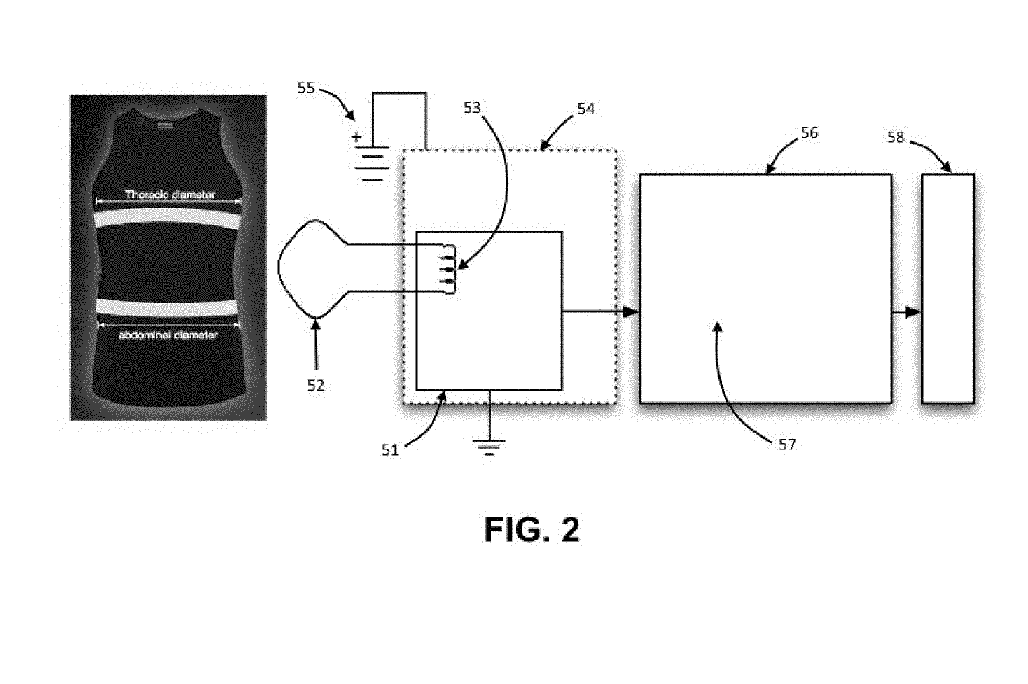

Preferably, the low resistivity impedance effort system of the invention comprises the use of a wire loop placed within the wearable garment. The impedance loop used is preferably a wire strategically placed in a textile guide incorporated into the garment or object fabric (as exemplary shown in FIG. 2). The garment loop 52 extends from one connector contact to another going around the torso of the wearer. In some embodiments, the two connectors are located at the garment effort belt 53 inside the Colpitts oscillator 51. The Colpitts oscillator 51 may further be controlled by an analog front end 54 for conditioning sampling and may be powered by a portable power source, such as a battery 55. The wearable device computes the statistics 57 such as breathing rate or breathing volume or tidal volume or the fractional inspiration time. In some embodiments, the wearable device comprises a central processing unit (CPU) 56 or is adapted to communicate with a CPU 56 to compute the statistics 57. In yet other embodiments, the statistics may be communicated through an application programming interface (API) 58.

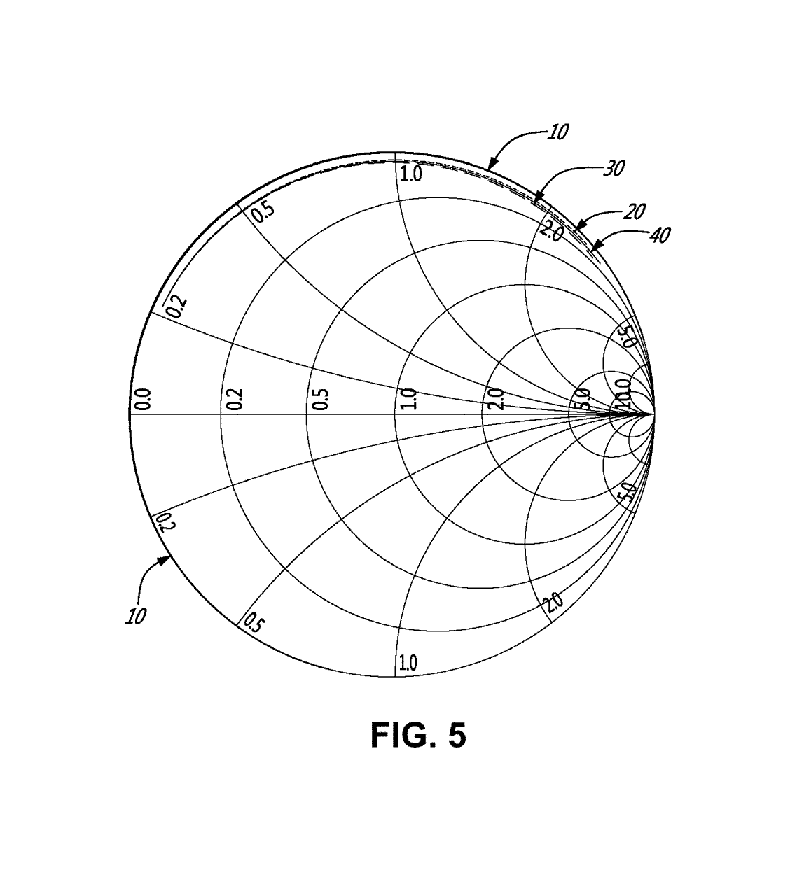

FIG. 5 shows a Smith chart result of the RIP sensor stimulated between 1 MHz and 15 MHz, of impedance of a garment using a Vectorial analyzer HP 8753 300 kHz-3.0 GHz [Canal 1 Ind. Att1=0 dB; Att2=0 dB; R/Z0 series: G/Y0 paral. Scale factor=1.00 U FS; IF=3.00 kHz; Z0=50.0]

The results of FIG. 5 are presented in the Table below:

Foper = 4.015 MHz

| Reference | | | | | |
|---|---|---|---|---|---|
| on FIG. 5 | Samples | L₀ | Z₀ | Xl | R |
| 10 | Body form with air | 1.88 μH | 47.38 | 47.35 | 1.61 |
| | (maximum diameter) | | | | |
| 20 | Body form with air | 1.89 μH | 47.65 | 47.72 | 1.64 |
| | (maximum diameter) | | | | |
| 30 | Human body | 1.95 μH | 49.24 | 49.21 | 1.74 |
| 40 | Same garment as 10 and 20 | 2.05 μH | 51.69 | 51.66 | 1.87 |
| | but with a human body | | | | |

[Minimal frequency = 1.00 MHz; Maximum frequency = 10.00 MHz; Electric delay = 0.000 s; dϕ = 0.00°; Sweeping = 100.00 ms; Type: VS Freq. Lin Mode: S11—Conversion = none]

The inductance variation due to movement of the electronic device, such as the RIP, is very small but more efficient. Movement of the body part produces Delta Inductance, then producing a delta frequency, then producing a delta amplitude, then producing n bit sampling. The Colpitts oscillator 51 in the frequency range from 1 MHz to 15 MHz is proven to be linear. FIG. 5 shows an excellent linearity with a resulting impedance around 2 micro Henry (μH).

To reduce power consumption further, the Colpitts oscillator 51 can be turned ON and OFF many times per second. Sufficient ON time is needed to be able to sample the frequency of the Colpitts oscillator 51.

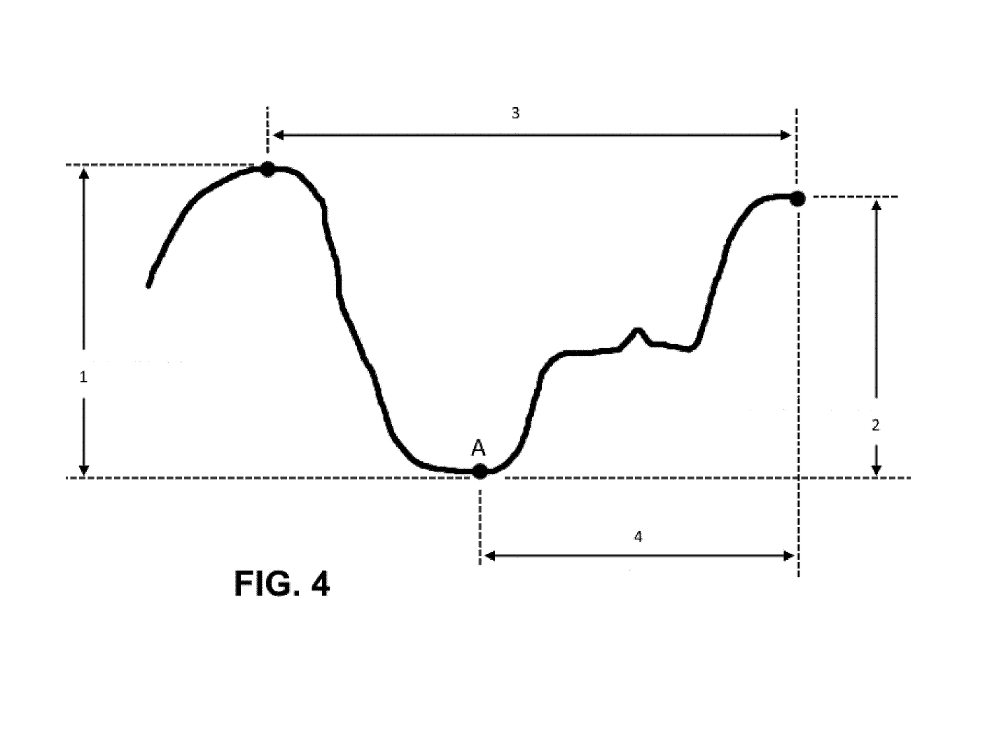

As described in FIG. 4, two criteria are considered to detect inspiration/expiration. One is the adaptive filter threshold (1, 2); the other is the eye closing (3, 4) (the inhibition period). In FIG. 4, an expiration is found when the condition (point A, minimum). It also applies to detection of inspiration but searching for maximum.

One example of adaptive Threshold resp is shown in FIG. 4, where:
25% of the average duration of the 4 last expirations
5≥Threshold resp≥50

One example of adaptive Eye_closing is also shown in FIG. 4, wherer:
25% of the average duration of the 4 last respiration (i.e. inspiration+expiration)
16≥Eye_closing≥256 (at 128 Hz, thus 0.125-2 s)

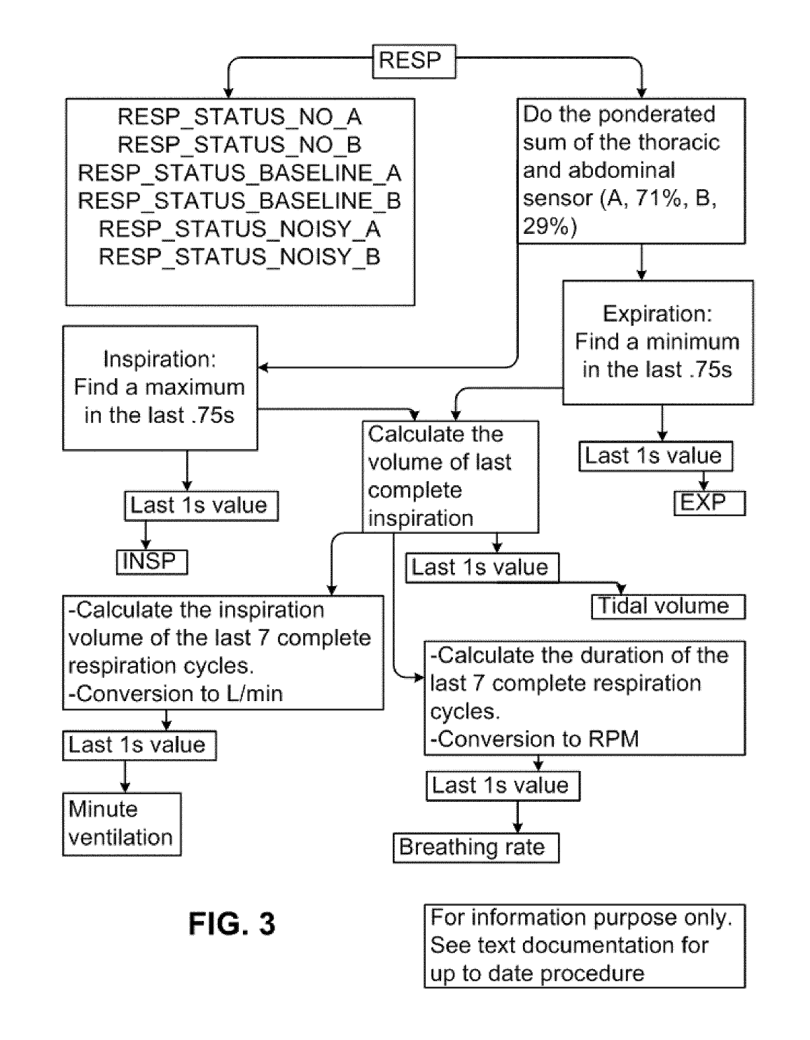

FIG. 3 shows an example of adaptive filtering with two RIP bands, using a weighted sum of the thoracic and abdominal signal for inspiration/expiration detection usage to extract minute ventilation, breathing rate, tidal volume and fractional inspiratory time (INSP: T inhale, EXP: T exhale). RESP is the sensing input coming from the Colpitts oscillator 51. Signal quality assessment is performed to validate input regarding the noise status of the sensor, its baseline linearity check and general status such connector connect/disconnect detection.

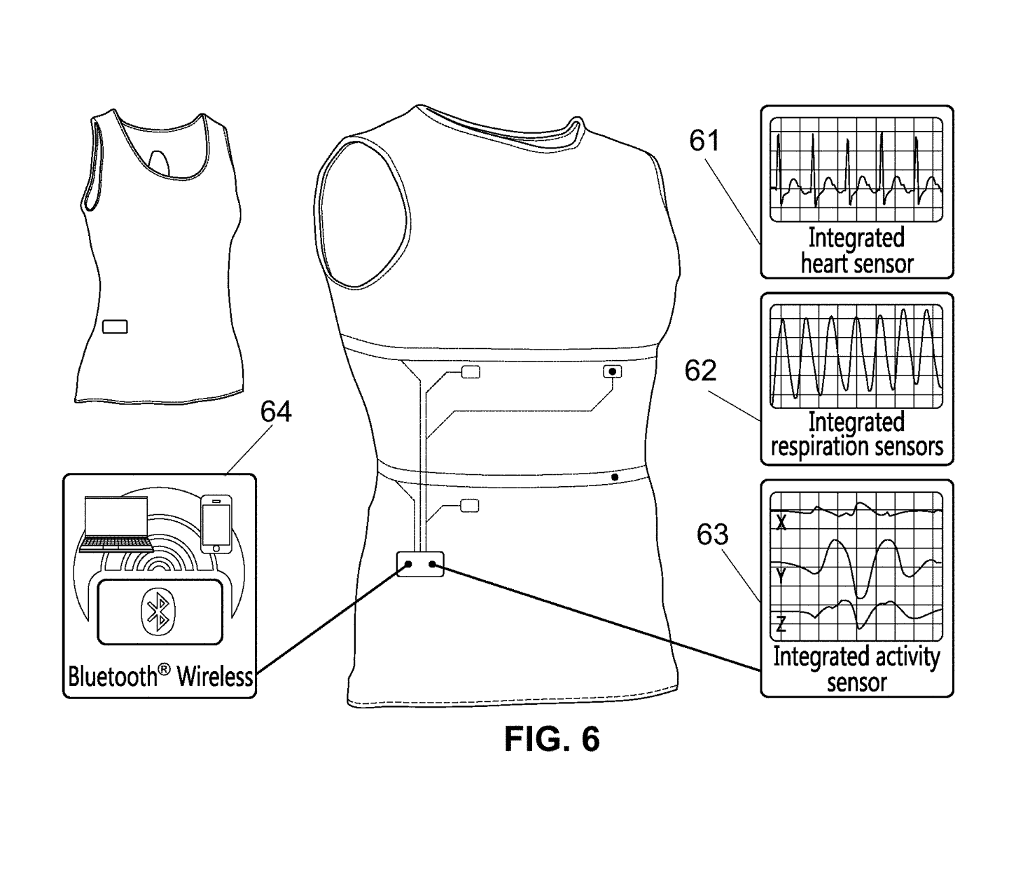

FIG. 6 shows an example of the RIP sensor integration in the wearable system. Different sensors may be integrated such integrated heart sensor 61, integrated respiration/breathing sensor 62, and integrated activity sensor 63. The sensors are normally passive and become active only once they are connected to the active electronic analog front end 54. Two RIP sensors are placed on a shirt, one on the torso one on the abdomen. Three textile electrodes are also placed, one differential input (ECG lead I) and one reference. All sensors electrical signal lines are interconnected through the connector to the small wireless apparatus 64. An apparatus comprising a 3-axis accelerometer motion sensor, local memory for data, processing capabilities to analyze data in real-time, and BLUETOOTH® communication capabilities, is used to communicate with smart phones and computers. the BLUETOOTH® wireless connection being a type of wireless technology for exchanging data over short distance. The data is processed and analyzed in the device in order to transmit only what is important to minimize power consumption. The smart phone and computer network connectivity make possible remote server communication, which can provide automatic physiological data analysis services and help with the interpretation of physiological signals.

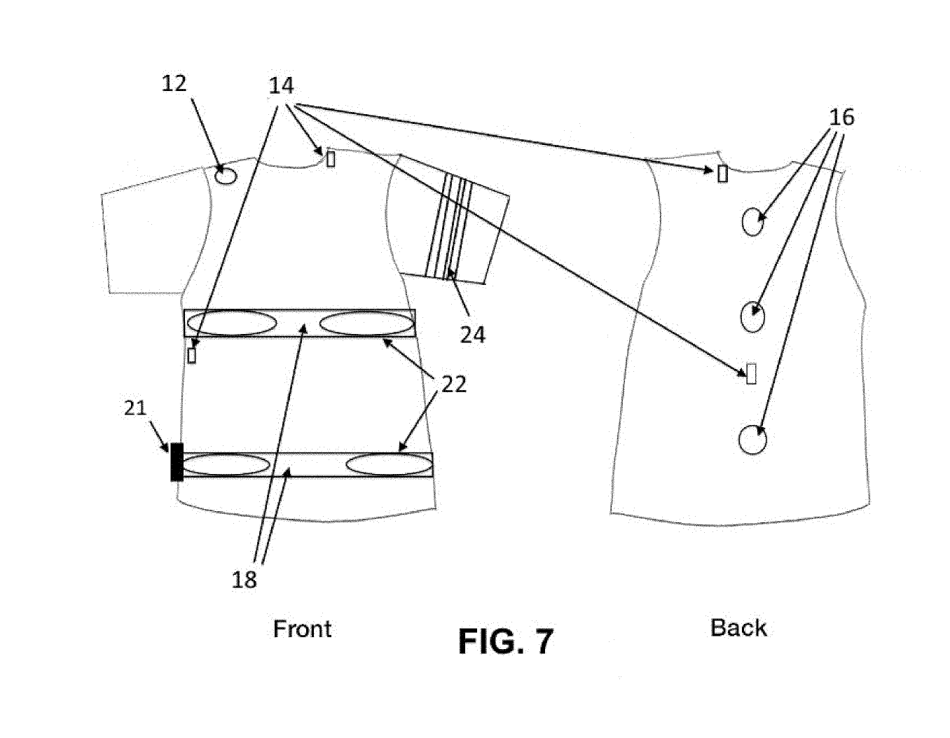

FIG. 7 is another wearable garment example where many more sensors are integrated into the fabric. For each sensor a different wiring technique can be used such as wires, knitted conductive fibers, laminated conductive textile, optic fiber and/or polymer. Sensors can be strategically placed to perform good quality biometric measurements. FIG. 7 shows a garment in accordance with one embodiment of the invention having two RIP band sensors 18, four textile electrodes ECG 22, a caught pressure sensor on the left arm 24, four temperature sensors 14, three position and orientation sensors 16, and an optical spectroscopy sensor 12. Other type of sensors such as galvanic skin response (GSR), stretch sensors for structural sensing and others can be used. The garment also comprises an electronic device 21, preferably a low-powered electronic device, for converting analog information measured by the Colpitts oscillator 51 into digital analyzable information.

### Example 1: Shirt for Men—Small Size

Duty cycle of 50%, with a time ON for the breathing circuit of 20 ms.

Oscillation frequency: 4.3 MHz

### Example 2: Shirt for Men—Large Size

Duty cycle of 50%, with a time ON for the breathing circuit of 20 ms.

Oscillation frequency: 5.4 MHz

The oscillation frequency varies between the two examples above due to the shirt's impedance with the wire length of different size.

The present invention also concerns a method for extracting physiological parameters of a person. The method comprises at least the followings steps:
a) providing a wearable garment, the garment fitting a body part of the person;
b) measuring at least one plethysmographic signal using at least one wire supported by or embedded into the garment, each wire forming a loop around the body part;
c) extracting the plethysmographic signal measured by each wire using a low-powered electronic device supported by the garment, the electronic device including a Colpitts oscillator 51 connected to each wire and having an optimal frequency band from 1 MHz to 15 MHz; and
d) converting analog information measured by the Colpitts oscillator 51 into digital analyzable information.

According to a preferred embodiment, the method may further comprise the step of connecting the Colpitts oscillator 51 to each wire using at least one connector embedded into the garment. As aforesaid, the number of connectors will depend on the number of wire loop to be connected to the electronic device.

According to a preferred embodiment, the method may further comprise the step of maintaining each wire in a predetermined position around the body portion using a guiding portion embedded into the garment.

According to a preferred embodiment, when the body portion is the torso of the person wearing the garment, the method may then comprise the steps of:
providing a first loop of said wires around a thoracic section of the torso;
providing a second loop of said wires around an abdominal section of the person; and
measuring a breathing frequency and/or frequency change of the person.

According to a preferred embodiment, the method may further comprise the step of making each wire extensible by using an extensible configuration of a conductive material.

According to an embodiment, the method may further comprise the step of powering the Colpitts oscillator 51 and low-powered electronic device using a power source. Preferably, the electricity power source is embedded into the garment. Preferably, the electricity power source may be a battery.

According to a further embodiment, the method may further comprise the step of turning on and off the Colpitts oscillator 51 a plurality of times per second according to a frequency sampling to extend a power life of the power source.

Preferably, in the method according to the present invention, the step of converting analog information into digital information further comprises the step of analyzing the information by applying at least one algorithm.

According to another embodiment, the method may further comprise the step of communicating the information from the electronic device to a smart phone or a computer using a wireless connection. The wireless connection may be a BLUETOOTH® connection, but other known wireless communications can be used. The BLUETOOTH® wireless connection being a type of wireless technology for exchanging data over short distance.

According to an embodiment, the method may further comprise the step of measuring body temperature, blood pressure and/or heart beat frequency using at least one sensor embedded into the garment and connected to the electronic device.

Preferably, the physiological parameters extracted by the application of the present method are breathing metrics such as, but not limited to respiratory rate, tidal volume, minute ventilation and fractional inspiratory time.

According to another embodiment, the method may further comprise the step of detecting and characterizing physical conditions such as, but not limited to talking, laughing, crying, hiccups, coughing, asthma, apnea, sleep apnea, stress related apnea, relaxation exercise, breathing cycle symmetry, and pulmonary diseases.

According to yet another embodiment, the method may further comprise the steps of detecting and characterizing heart activities such as but not limited to heart rate, body movements and activities, such as walking and running.

In some embodiments, the wearable system 100 may be calibrated using external data in order to provide exact or at least precise oscillation measurements. The external data may comprise a plurality of metrics, such as the breathing rate, the minute ventilation and/or tidal volume. The followings paragraphs exemplify the usage of such metrics.

The breathing rate is the speed at which a user completes a full breathing cycle, thus including inspiration and expiration periods. The breathing rate is typically defined as the number of respirations per minute (rpm). The breathing rate is typically not affected by the volume of the respiration of a user. When a user is at rest, the breathing rate typically ranges between 12 to 16 rpm. When a user is sleeping, the breathing rate may be as low as 6 rpm. When a user is executing high intensity exercise, the breathing rate may reach more than 70 rpm. Other factor causing variation of the breathing rate may comprise voluntary effort, stress, anxiety, temperature and/or altitude.

Amongst other, a user may monitor his breathing rate to be aware of the efficiency of his breathing, to store or analyze personal values and the evolution of such values with regard to time and/or activities or to identify a respiratory problem (ex. sleep apnea).

Another metric that may be monitored is the minute ventilation. The breathing volume is typically represented as being the volume of air inspired by a user that transited in the lungs. Typically, the breathing volume is represented as a number of liters per minute (L/min). The minute ventilation can be defined as the total volume inspired by a user during a period of time, such as during one minute. The normal breathing volume is typically between 6-8 L/min for a normal person. When a user is sleeping, the breathing volume may be as low as 3 L/min. During high intensity activity, such as athletes, the breathing volume may be as high as 160 L/min.

The breathing rate and breathing volume are generally used to find correlation with energy consumption and stress of a user. The monitoring of the breathing volume may be used to monitor hyperventilation of the body of the user, such as prior to a stressful event such as a competition.

The minute ventilation metric may be associated with abnormal conditions such as hyperventilation or hypoventilation.

Hyperventilation is generally understood as describing a minute ventilation being higher than what is physiologically suitable. Hyperventilation may be voluntary or involuntary and occurs when a CO2 output of a user (through alveolar ventilation) exceeds the production of CO2 of the body of the user. Hyperventilation may be a consequence of medical conditions, of voluntary hyperventilation, of high altitude, of emotional or physical stimuli. Other factors may cause hyperventilation, such as stress and anxiety, also known as hyperventilation syndrome.

Hyperventilation may also occur when a user is exercising at a VO2max intensity. At such level, the user is unable to produce energy through aerobic respiration (with oxygen). Thus, the user loses breathing control and hyperventilate in an effort to catch more oxygen.

Hypoventilation is generally understood as describing a minute ventilation being lower than what is physiologically suitable. Hypoventilation may occur through voluntary or involuntary actions and may occur when minute ventilation is too low to allow appropriate gas exchanges. In other words, production of CO2 of the user exceeds CO2 output in the lungs and leads to an increase in of CO2 concentration, also called respiratory acidosis. Hypoventilation may be a consequence of medical conditions, of holding breath, of chronic mountain sickness or of drugs usage.

Another metric that may be monitored is tidal volume. The tidal volume is generally the volume of air (usually represented in liters) of one inspiration or expiration of a user. A healthy, young adult at rest generally has a tidal volume of approximately 500 mL per inspiration or 7 mL/kg of body mass. During a forced inspiration and expiration, the tidal volume of a user is, on average, 4.8 L for men and 3.7 L for women.

The present system may monitor tidal volume to compute minute ventilation. Based on tidal volume and breathing rate, the system is configured to estimate minute ventilation. The tidal volume may be monitored in different circumstances, such as at rest (i.e. relaxation, recovery) or during exercise (i.e. breathing control). The tidal volume may be monitored in a clinical environment, such as for detecting respiratory problems of a user, such as but not limited to asthma.

In some embodiments, a calibration method to adjust the respiration coefficient parameter kᵣₑₛₚ based on external data is provided. To execute the calibration method, a user typically dons or puts on the garment. The electronic device connected to the garment is in communication with a storage device or data unit. In typical embodiment, the electronic device 54 comprises a oscillator and preferably a Colpitts oscillator 51 connected to each wire loop 52 of the garment. In a preferred embodiment, the storage device is configured to record or save data.

The method further comprises measuring vital capacity (VC) or lung capacity of a user. Typically, a user expires air in an electronic device to measure lung capacity, such as but not limited to a spirometer. Typically, the method comprises taking a plurality of measurements of the lung capacity.

In such an embodiment, the plurality of measurements of VC are used to calculate the respiration coefficient parameter kᵣₑₛₚ of each user or person. Typically, the kᵣₑₛₚ is calculated using a linear method or linear regression on a series of reference or base measurement values. As an example, a constant coefficient of the reference measurements function associated with the average of the measurements may be used to calculate kᵣₑₛₚ. The reference measurements are preferably obtained with a golden standard external apparel, such a spirometer. Understandably, the reference measurements may be obtained. from experimentation on typical categories of subjects or using any known method to capture such reference measurements values. Understandably, any other known method using reference values may used without departing from the scope of the present invention.

The measurements of the lung capacity are saved in the electronic device. In some embodiments using a spirometer, the measurements may be communicated from the spirometer to the storage device using any data communication means, such as a wireless data communication link.

The method further comprises the data storage communicating the measurements to the electronic device 52 through any communication means. At this stage, the data storage unit may be disconnected from the electronic device 54 to avoid acquiring new measurement data before the calibration method is completed.

The method further comprises inputting the measurements data into a system configured to calculate VC_{BIOM} at the time of acquisition of the measurements. The measurement data typically comprises the time at which each measurement was captured or taken, the vital capacity (VC) value of each measurement, such as number of liters.

In some embodiments, the method further comprises calculating VC_{BIOM} at the time of acquisition of the measurements using the following equations:

VC_{ref}=VC*kᵣₑₛₚ

VE_{calibrated}(t)=VE_{raw}(t)*kᵣₑₛₚ
where VE is the minute ventilation, VC is the vital capacity value, kᵣₑₛₚ is the respiration coefficient parameter.

When the system 100 is calibrated, the plethysmographic signal extracted by the oscillator 51 is multiplied by the respiration coefficient parameter calculated during the calibration method to produce a calibrated plethysmographic signal.

In some embodiments, as the temperature of the electronic device 54 increases, the detection of inspiration/expiration may become faulty or a drift effect (non-linear effect) may be observed. To correct the drift effect, a temperature sensor or thermometer may be connected to the wearable system or the electronic device. The temperature sensor 70 is generally configured to detect or measure surrounding temperature and to produce a signal or a value as a function of the detected surrounding temperature. In a typical embodiment, the temperature sensor measures or detects the temperature value at a predetermined frequency.

In a preferred embodiment, the temperature sensor is configured to sample the temperature value minimally at 1 Hz,

In some embodiment, the electronic device 54 may further be configured to filter or measured temperature signal 72 or the calculate a corrected temperature signal to, as an example, reduce the noise in the temperature signal. Understandably, in such embodiments, any known method to filter the temperature signal may be used.

The wearable system is configured to correct the drift effect of the respiration signal due to temperature variation. The wearable system thus is the uncorrected or raw respiration signal and use a correction method to produce a corrected respiration signal.

In a typical embodiment, the temperature sensor 70 has a precision or increment of about 0.1° C. between 20° C. and 40° C. In some other embodiments, the precision may range from ±0.05° C. (typ) or ±0.13° C. (max) from 20° C. to 42° C. or ±0.2° C. (max) from −20° C. to 90° C. Understandably, any other precisions level of the temperature sensor allowing enough precision to measure temperature variation of the electronic device 52 may be used.

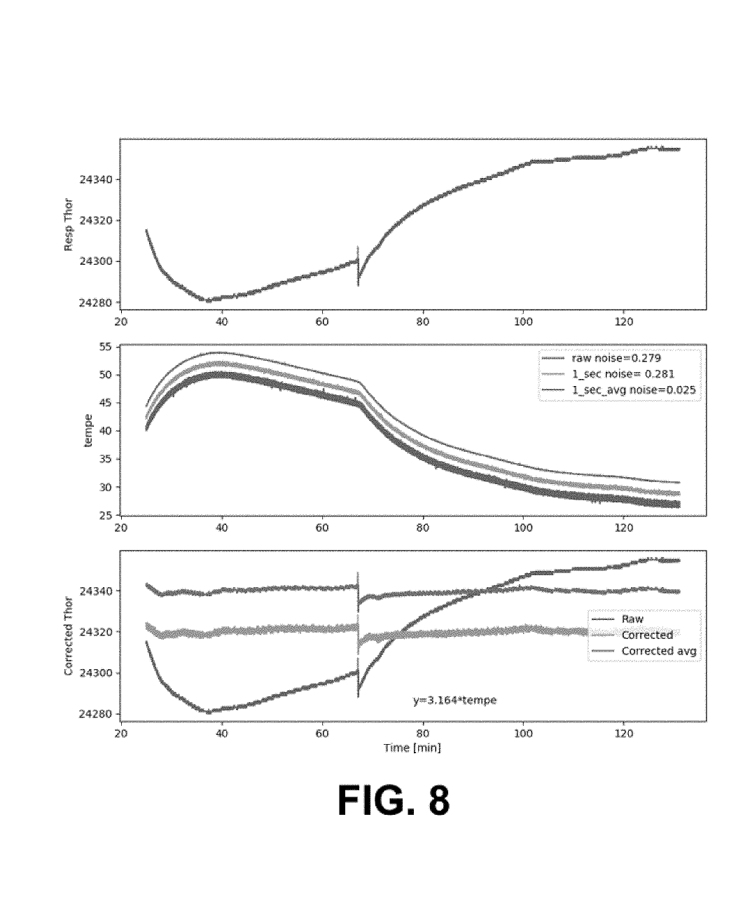

Now referring to FIG. 8, different graphs of results showing an increase in temperature as the oscillation frequency of the sensor decreases are illustrated. Based on experimental results (not shown), the coefficient to correct the drift generated by the variation of temperature for thoracic and abdominal signals independently is calculated following the below equations:

Thor_{corrected}(t)=Thor(t)+(tempe(t)−tempe_{ref})k_{freq-thor}

Abdo_{corrected}(t)=Abdo(t)+(tempe(t)−tempe_{ref})*k_{freq-abdo}
Where: Tempe_{ref} is a reference temperature, such as the temperature in Celsius after a predetermined duration after the start of the device and where k_{freq} is a factor which varies based on the oscillation frequency of the wearable system. In such an example, the k_{freq-thor}=3.3 and k_{freq-abdo}=1.1.

Understandably, Tempe_{ref} may be adapted to any value depending on the implementation of the system, such as if the calculation is executed on a remote server or directly on the electronic device.

The equation may be generalized to any body portion of the user which may change shape or volume:

BodyPortion_{corrected}(t)=BodyPortion(t)+(tempe(t)−tempe_{ref})*k_{freq-bodyPortion}

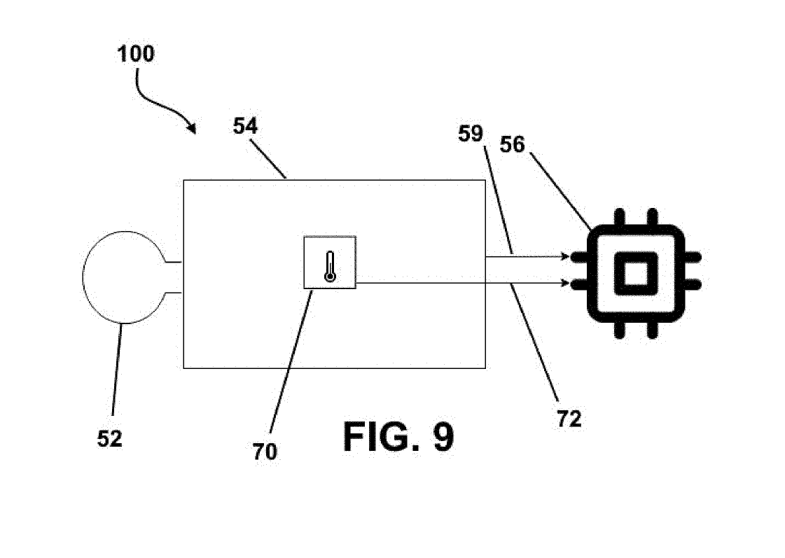

Now referring to FIG. 9, an embodiment of a wearable system 100 comprising a temperature sensor 70 is shown. The wearable system 100 comprises a loop 52 embedded in a garment. The loop 52 is connected to an electronic sensor circuit 54 calculating or extracting oscillation frequency 59 of the loop 52. In such an embodiment, the electronic sensor circuit 54 further comprises a thermometer or temperature sensor 70 adapted to detect and/or measure the surrounding temperature and preferably the temperature of the electronic sensor circuit 54. The temperature sensor is preferably in communication with a CPU 56 and transmits the temperature value or signal 72 at a predetermined frequency. The electronic sensor circuit 54 is in communication with the CPU 56 and transmits the oscillation frequency 59 of the loop 52.

The CPU 56 is configured to execute instructions to calculate a corrected oscillation frequency based on the received oscillation frequency 59 and on the temperature value 72.

Understandably, the calculation of the corrected oscillation frequency may be executed in the electronic device 54 having a CPU 56 or may be calculated on an external computerized device, such as a mobile phone, a table, a computer, a smart watch or any other type of computerized device capable of being in communication with the electronic device 54.

In embodiments where the calculation is executed on an external device, the raw or uncorrected oscillation frequency may be communicated from the electronic device 54 to the external device through a network, preferably a wireless network. In such embodiments, upon calculating the corrected oscillation frequency, the external device may be capable of communicating the corrected oscillation frequency to the electronic device 54 or to any other computerized device through the network.

A method for correcting drifting due to temperature variation of the electronic sensor device 54 is further disclosed. The method comprises retrieving or calculating the oscillation frequency of the garment loop 52 using an electronic circuit 54, measuring the temperature 72 of the electronic circuit 54 and calculating a corrected oscillation frequency based on the oscillation frequency 59 and on the temperature value 72.

The method may further comprise communicating the oscillation frequency 59 and the temperature value to a controller or CPU 56 and the CPU 56 calculating the corrected oscillation frequency.

The method may further comprise calculating the oscillation frequency using the following equations:

BodyPortion_{corrected}(t)=BodyPortion(t)+(tempe(t)−tempe_{ref})*k_{freq-bodyPortion}

The scope of the claims should not be limited by the preferred embodiments set forth in the examples, but should be given the broadest interpretation consistent with the description as a whole.

What is claimed is:

1. A wearable system for extracting physiological parameters of a person by measuring at least one plethysmographic signal, the system comprising:
a wearable garment configured to fit a body portion of the person;
at least one wire supported by or embedded into the garment, each wire configured to form a loop around the body part when the person wears the garment for measuring a plethysmographic signal;
an electronic device supported by or fixed on the garment and including a Colpitts oscillator connected to each wire loop;
a temperature sensor configured to measure temperature of the electronic device;
wherein the oscillator is configured to extract the plethysmographic signal measured by each wire at a predetermined frequency, the electronic device converting analog information measured by the oscillator into digital analyzable information;
wherein the measured temperature of the electronic device is used to calculate a corrected plethysmographic signal from the extracted plethysmographic signal.

2. The system of claim 1, the system further comprising at least one connector embedded into the wearable garment for connecting the oscillator to each wire loop.

3. The system of claim 1, wherein the wearable garment comprises at least one guiding portion embedded into the garment, each guiding portion comprising a cavity to receive and maintain one of said at least one wire in a predetermined position around the body portion.

4. The system of claim 1, wherein the body portion is the torso of the person wearing the wearable garment, the system then comprising a first loop of said wires configured to be placed around a thoracic section of the torso and a second loop of said wires configured to be placed around an abdominal section of the person; the first and second loops being configured to measure a breathing frequency and/or a frequency change of the person.

5. The system of claim 1, wherein each wire loop is constructed using a conductive material in a configuration that makes the wearable garment extensible.

6. The system of claim 1, further comprising a power source for powering the oscillator and the electronic device.

7. The system of claim 6, wherein the oscillator is adapted to be turned on and off a plurality of times per second according to a frequency sampling to extend a power life of the power source.

8. The system of claim 1, wherein the electronic device is a digital processing device comprising a processing unit configured to execute instructions for converting analog information into digital information by applying at least one algorithm to analyze the information.

9. The system of claim 1, wherein the electronic device is in communication with a smart phone or a computer using a wireless connection.

10. The system of claim 1, further comprising at least one sensor for measuring body temperature, blood pressure and/or heart beat frequency.

11. The system of claim 1, wherein the physiological parameters extracted by the system are one or more breathing metrics selected from the group consisting of respiratory rate, tidal volume, minute ventilation and fractional inspiratory time.

12. The system of claim 1, wherein the wearable system comprises one or more sensors to measure one or more metrics to detect and characterize physical conditions selected from the group consisting of talking, laughing, crying, hiccups, coughing, asthma, apnea, sleep apnea, stress related apnea, relaxation exercise, breathing cycle symmetry, and pulmonary diseases.

13. The system of claim 1, wherein the wearable system comprises one or more sensors to measure one or more metrics to detect and characterize heart activities selected from the group consisting of heart rate, body movements and body activities.

14. The system of claim 1, the oscillator being a Colpitts oscillator.

15. The system of claim 14, the Colpitts oscillator having an optimal frequency band from 1 MHz to 15 MHz for extracting the plethysmographic signal.

16. The system of claim 15, wherein the frequency of the Colpitts oscillator is about 4.3 MHz.

17. The system of claim 16, wherein the frequency of the Colpitts oscillator is about 5.4 MHz.

18. The system of claim 1, the at least one wire having an inductance varying between 76 nH and 4.4 μH.

19. The system of claim 1, the temperature sensor having a precision of about 0.1 degree Celsius.

20. A method for extracting physiological parameters of a person, the method comprising the steps of:
providing a wearable garment, the wearable garment fitting a body portion of the person;
measuring at least one plethysmographic signal using at least one wire supported by or embedded into the garment, each wire forming a loop around the body part;
extracting the plethysmographic signal measured by each wire using an electronic device supported by the garment, the electronic device comprising a oscillator connected to each wire;
converting analog information measured by the Colpitts oscillator into digital analyzable information;
measuring the temperature of the electronic device;
calculating a corrected plethysmographic signal from extracted plethysmographic signal using the measured temperature of the electronic device.

21. The method of claim 20, the calculation of the corrected plethysmographic signal at a body portion having a variable volume of the person using the following equation:
BodyPortion_{corrected}(t)=BodyPortion(t)+(tempe(t)−tempe_{ref})*k_{freq-bodyPortion}
Where: Tempe_{ref} is a reference temperature and where k_{freq} is a factor which varies based on the frequency of oscillation.

22. The method of claim 21, the body portion being selected in any of the thoracic portion, the abdominal portion or arm portion of the person.

23. A method for calibrating extraction of physiological parameters of a person, the method comprising:
the person wearing a garment comprising at least one wire supported by or embedded into the garment, each wire being configured to form a loop around the body part when the person wears the garment for measuring a plethysmographic signal;
extracting the plethysmographic signal measured by each wire using an electronic device supported by the garment, the electronic device comprising a oscillator connected to each wire;
measuring data regarding vital capacity of the person by taking a plurality of measurements;
storing the vital capacity measurements in a data storage in communication with the electronic device;
calculating vital capacity coefficient of the person at the time of acquisition of the measurements using the vital capacity measurements;
calculating a respiration coefficient parameter based on the vital coefficient of the person;
calculating a corrected plethysmographic signal by multiplying by the respiration coefficient parameter with the extracted plethysmographic signal.

24. The method for calibrating extraction of claim 23, the measuring of the vital capacity of the person using a spirometer.

25. The method for calibrating extraction of claim 23, the data regarding vital capacity comprising any one of breathing rate, minute ventilation or tidal volume.

26. The method for calibrating extraction of claim 23, disconnecting the data storage from the electronic device prior to the calculating vital capacity coefficient.

27. The method for calibrating extraction of claim 23, the calculation of the corrected plethysmographic signal using the following equations:
VC_{ref}=VC*kᵣₑₛₚ
VE_{calibrated}(t)=VE_{raw}(t)*kᵣₑₛₚ
where VE is the minute ventilation, VC is the vital capacity value, kᵣₑₛₚ is the respiration coefficient parameter.
